(19) United States
(12) Patent Application Publication
(10) Pub. No.: US 2014/0051081 A1
(43) Pub. Date: Feb. 20, 2014

(54) METHOD OF SCREENING FOR CANCER BY DETECTING MUTATIONS IN THE DELTA-CATENIN CODING REGION

(71) Applicants: Qun Lu, Greenville, NC (US); Yan-Hua Chen, Greenville, NC (US)

(72) Inventors: Qun Lu, Greenville, NC (US); Yan-Hua Chen, Greenville, NC (US)

(73) Assignee: EAST CAROLINA UNIVERSITY, Greenville, NC (US)

(21) Appl. No.: 14/007,744

(22) PCT Filed: Mar. 29, 2012
(86) PCT No.: PCT/US12/31110

§ 371 Date: Nov. 6, 2013

Related U.S. Application Data
(60) Provisional application No. 61/468,726, filed on Mar. 29, 2011.

(51) Int. Cl. C12Q 1/68

(52) U.S. Cl. CPC C12Q 1/6886
USPC 435/6.11

(57) ABSTRACT

Aspects of the present invention provide methods such as screening for risk of cancer, detecting cancer and/or determining treatment for cancer in a subject including detecting the presence or absence of at least one mutation in the delta-catenin coding region. Aspects of the present invention also provide kits for carrying out the methods described herein.

### GOVERNMENT SUPPORT

This invention was made with government support under grant numbers RO1-CA111891 from the National Cancer Institute and RO3-ES016888 from the National Institute for Environmental Health Sciences. The US Government has certain rights to this invention.

### BACKGROUND OF THE INVENTION

Prostate cancer is a major cause of death among men in Western countries. The current protocol for detection of this cancer involves testing for prostate-specific antigen (PSA) levels. If PSA levels are found to be high (4 ng/ml), a tissue biopsy is performed. Unfortunately, PSA testing is limited by the fact that it lacks sensitivity and it does not distinguish between prostate cancer and benign prostate hyperplasia. As a result, many men either are not identified as having the disease or because of false positive tests are subjected to the invasive tissue biopsies when they do not have the disease. A much more specific and less invasive diagnostic test is needed for early detection of this disease.

Delta-catenin presents itself as an improved alternative to the PSA/biopsy tests currently utilized for prostate cancer detection. Delta-catenin was first identified and patented (U.S. Pat. No. 6,258,929) as a neurospecific protein, alternatively named ALARM. At the time, the protein was believed to be expressed almost exclusively in brain tissue. However, Burger, et al. (Int. J. Cancer 100, 228-237 (2002)) subsequently found the messenger RNA for delta-catenin to be expressed in prostate cancer tumors with the delta-catenin transcripts being localized to the glandular secretory cells. Unlike PSA, delta-catenin was capable of distinguishing between prostate cancer and benign prostate hyperplasia. Burger et al. noted a possible diagnostic role for delta-catenin in prostate cancer detection. However, they also pointed out that a significant difficulty remained in development of this tool since delta-catenin had only been detected in glandular secretory epithelial cells in prostate tissues and had not been found in prostate stroma or bodily fluids, such as serum or urine.

### SUMMARY OF THE INVENTION

A first aspect of the present invention is a method for screening for cancer or risk of cancer (“risk” including cancer aggressiveness or risk of an aggressive) in a subject comprising the steps of: detecting the presence or absence of at least one mutation in the delta-catenin coding region or 3′ untranslated region in a biological sample from said subject; the presence of said mutation or an increased frequency of mutation (e.g., as compared to an unafflicted subject or a population or group of unafflicted subjects) indicating said subject is afflicted with or at least at risk of developing cancer.

It will be appreciated that the instant invention concerns, among other things, detecting increased incidences of nucleotide polymorphic changes in the delta-catenin coding region or 3′ untranslated region in a biological sample from a subject, and determining or indicating an increased risk of or affliction with cancer from the increased incidence of such changes detected (as compared to subjects not afflicted with that cancer or not at increased risk).

Mutations that may be detected include, but are not limited to, delta-catenenin coding region or 3′ untranslated region mutations set forth in Table 1 and FIG. 1 below.

Subjects may be male or female, and cancers to be screened include but are not limited to lung, breast, colon, prostate, esophageal, ovarian, pancreatic, adrenal, skin cancer and leukemia. In some embodiments the subject is male, and said cancer is prostate cancer.

Another aspect of the invention is, in a method for screening for cancer in a subject by detecting the presence of a cadherin, prostate specific antigen, and/or p120 cancer biomarker in said subject, the improvement comprising the steps of: detecting the presence or absence of at least one mutation in the delta-catenin coding region or 3′ untranslated region in a biological sample from said subject; the presence of said mutation or an increased frequency of mutation indicating said subject is afflicted with or at least at risk of developing cancer. Subjects, mutations, and cancers may be as described above.

A further aspect of the invention is the use of a means of detecting the presence or absence of mutation in the delta-catenin gene coding region or 3′ untranslated region in a biological sample from a subject for carrying out a method as described above,

A further aspect of the invention is a kit comprising a means of detecting the presence or absence of mutation in the delta-catenin gene coding region or 3′ untranslated region in a biological sample from a subject for carrying out a method as described above, the kit optionally including instructions for carrying out a method as described above.

The foregoing and other objects and aspects of the present invention are explained in greater detail in the drawings herein and the specification set forth below.

### BRIEF DESCRIPTION OF THE DRAWINGS

FIG. 1 depicts a diagram of particular CTNND2 coding region mutations associated with prostate cancer.

### DETAILED DESCRIPTION

The present invention is explained in greater detail below. This description is not intended to be a detailed catalog of all the different ways in which the invention may be implemented, or all the features that may be added to the instant invention.

For example, features illustrated with respect to one embodiment may be incorporated into other embodiments, and features illustrated with respect to a particular embodiment may be deleted from that embodiment. In addition, numerous variations and additions to the various embodiments suggested herein will be apparent to those skilled in the art in light of the instant disclosure which does not depart from the instant invention. Hence, the following specification is intended to illustrate some particular embodiments of the invention, and not to exhaustively specify all permutations, combinations and variations thereof.

Unless otherwise defined, all technical and scientific terms used herein have the same meaning as commonly understood by one of ordinary skill in the art to which this invention belongs. The terminology used in the description of the invention herein is for the purpose of describing particular embodiments only and is not intended to be limiting of the invention.

“Cancer” as used here includes but is not limited to brain, esophageal, lung, breast, colon, ovarian, rectal, testicular, prostate, urethral, bladder, liver, genitourinary, gastrointestinal, pancreatic, adrenal, skin cancer, adenocarcinomas, hyperproliferative disorders or leukemia.

“Subject” as used herein refers to animal subjects, particularly mammalian subjects, including but not limited to, humans, non-human primates, dogs, cats, rabbits, goats, horses, pigs, and cattle. The subject may be a male subject or a female subject and may be of all ages including infant, juvenile, adolescent and adult subject.

“Biological sample” as used herein may be any sample from the subject containing a nucleic acid of the subject to be analyzed for the mutation of interest, or a protein of the subject to be analyzed for the mutation of interest. Tissue samples (from afflicted or unafflicted organs) may be used, and/or fluid samples or body fluid samples.

“Fluid sample” or “body fluid sample” as used herein includes but is not limited to blood samples, seminal fluid, urine, bronchial lavage or aspirates, and aspirates or fine needle aspirates from suspected afflicted organs such as the prostate in a subject.

“Blood sample” as used here refers to whole blood, blood plasma, blood serum or any fraction thereof, so long as the fraction contains (in subject with cancer) delta-catenin as described herein.

The disclosures of all US Patent references cited herein are to be incorporated by reference herein in their entirety.

The present invention is, as noted above, drawn to methods for detection of cancer utilizing delta-catenin. Detection may be for diagnostic or prognostic purposes, may be for general screening purposes, may be for targeting cancer in chemotherapy, or may be for the purpose of determining if a subject is at risk of developing cancer, confirm a diagnosis, indicate the reoccurrence of cancer, etc.

As noted above, the subject may be a human subject, or an animal subject for veterinary or drug screening or development purposes, with examples of suitable animal subjects including but not limited to dogs, cats, rabbits, goats, horses, pigs, cattle, etc. The subjects may be a male subject or a female subject; the subject may be of any age including infant, juvenile, adolescent and adult subjects.

The present invention may be used to detect any type of cancer, including but not limited to brain, esophageal, lung, breast, colon, ovarian, rectal, testicular, prostate, urethral, bladder, liver, genitourinary, gastrointestinal, pancreatic, adrenal, skin cancer, adenocarcinomas, hyperproliferative disorders or leukemia. In some embodiments of the invention, the cancer to be detected is prostate cancer.

In an embodiment, the present invention screens for the risk of cancer in a subject by detecting the presence or absence of at least one mutation in the delta-catenin coding region of 3′ untranslated region in a biological sample from said subject. In particular embodiments, the present invention screens for the presence of a mutation or mutations in the delta-catenin coding region, wherein said mutation or mutations is located in exon 10, exon 13, exon 14, exon 15, exon 16, exon 17, exon 18, exon 19, exon 20, exon 21 and/or exon 22 of delta-catenin coding sequence. In a particular embodiment, the mutation or mutations is located in exon 10, exon 13, exon 14, exon 19, exon 20, exon 21 and/or exon 22 of delta-catenin coding sequence, and wherein the presence of said mutation in said subject is an indication that said subject is afflicted with or at least at risk of developing cancer.

δ-Catenin (NPRAP/Neurojungin; gene designation: CTNND2, NCBI RefSeq Accession No. NM_{—}001332.2 (mRNA), GenBank Accession No. EU350953 (complete cds)) is a unique β-catenin/armadillo domain-containing p120^{ctn} subfamily protein (NCBI RefSeq Accession No. NP_{—}001323.1) in that it is primarily expressed in the central nervous system. However, δ-catenin is upregulated in over 80% of human prostatic adenocarcinomas. Analyses of δ-catenin CpG islands in the promoter region in benign and prostate cancer specimens revealed no significant hyper- or hypomethylation. Real time PCR analyses found no evidence of gene amplification in δ-catenin. However, we have observed an increased incidence of mutations in the coding region or 3′ untranslated region of the δ-catenin gene when compared to that of benign prostatic tissue specimens as well as to that of peripheral blood samples of normal control subjects.

In a further embodiment of the invention, the mutation or mutations in the delta-catenin coding region are single nucleotide mutations. The single nucleotide mutation may be a substitution of a nucleotide base or the deletion of a nucleotide base. In another embodiment, the mutation or mutations may be a deletion or insertion of more than one nucleotide. In particular embodiments, the mutation or mutations is selected from (in reference to delta-catenin mRNA from the start ATG codon) the group consisting of 1671 G to A; 2256 G to A; 2300 T to G; 2424 A to C; 2432 G to T; 2447 A deleted; 2447 A to G; 2448, 2449 GA deleted; 3095 C to A; 3285 C to T; 3285 insert TA; 3285 C to G; 3287 insert GA; 3347 delete A; 3421 miss T; 3516 C to T; 3673 G to T; 3673 delete G; 3674 T to G; 3674 delete T; 3675 G to T; 3674 T to G; 3676 T to G; and 3677 delete G, or combinations thereof. In more particular embodiments, the mutation or mutations is selected from the group consisting of 1671 G to A; 2256 G to A; 2300 T to G; 3095 C to A; 3285 insert TA; 3285 C to G; 3347 delete A; 3516 C to T; 3673 delete G; 3674 delete T; and 3674 T to G, or combinations thereof.

In a further embodiment of the invention, the at least one mutation results in the expression of a mutated form of the delta-catenin protein. In a more particular embodiment, the mutated form of the delta-catenin protein is a truncated form of the protein and has a deletion at the carboxy terminus. The deletion at the carboxy terminus may be at least 5, 10, 15 or 20 or more consecutive carboxy-terminal amino acids deleted. In a particular embodiment, the deletion of the carboxy-terminal starts at amino acid 1113 of the delta-catenin protein sequence. In another particular embodiment, the deletion of the carboxy-terminal starts at amino acid 1212 of the delta-catenin protein sequence.

The screen for the risk of cancer of the present invention may include, in addition to the detection of the presence or absence of at least one mutation in the delta-catenin coding region of 3′ untranslated region in a biological sample from said subject, may include the screen for the presence of one or more additional biomarkers, wherein the presence of said biomarker in addition to the presence the at least one mutation is an indication that said subject is afflicted with or at least at risk of developing cancer.

The screening method of the present method may use any suitable method within the understanding of one of skill in the art to determine the presence or absence of a mutation within a nucleic acid sequence. In a particular embodiment, the screening method may involve the amplification of nucleic acid sequences.

Amplification of nucleic acids may be carried out by any suitable technique, including but not limited to polymerase chain reaction (including, for RNA amplification, reverse-transcriptase polymerase chain reaction), ligase chain reaction, strand displacement amplification, transcription-based amplification (see D. Kwoh et al., Proc. Natl. Acad. Sci. USA 86, 1173-1177 (1989)), self-sustained sequence replication (or “3SR”) (see J. Guatelli et al., Proc. Natl. Acad. Sci. USA 87, 1874-1878 (1990)), the Q.beta. replicase system (see P. Lizardi et al., Biotechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra). The bases incorporated into the amplification product may be natural or modified bases (modified before or after amplification), and the bases may be selected to optimize subsequent electrochemical detection steps. Techniques for amplification are known and described in, among other things, U.S. Pat. Nos. 4,683,195; 4,683,202; 4,800,159; and 4,965,188; G. Walker et al., Proc. Natl. Acad. Sci. USA 89, 392-396 (1992); G. Walker et al., Nucleic Acids Res, 20, 1691-1696 (1992); R. Weiss, Science 254, 1292 (1991).

A biological sample may be a cell sample, with an intervening culturing step being performed between the time the cell sample is collected from the subject and the immunoassay is carried out on the biological sample. In some embodiments, a sample is obtained by using rectal massage to release some cells into the urine and then collect the urine to perform PCR; in some embodiments void urine is collected and cell debris spun down therein to perform PCR; in some embodiments needle biopsy is carried out to obtain a tissue sample on which PCR is performed.

A biological sample may also be a cell, cell debris, or stroma sample, with an intervening step being performed before the sample is collected from the subject to enhance the sensitivities of the assay.

In general, the step of detecting the polymorphism of interest may be carried out by collecting a biological sample containing DNA from the subject, and then determining the presence or absence of DNA containing the polymorphism of interest in the biological sample. Any biological sample which contains the DNA of that subject may be employed, including tissue samples and blood samples, with blood cells being a particularly convenient source. The nucleotide sequence of the human delta-catenin gene is known and suitable probes, restriction enzyme digestion techniques, or other means of detecting the polymorphism may be implemented based on this known sequence in accordance with standard techniques. See, e.g., U.S. Pat. Nos. 6,027,896 and 5,767,248 to A. Roses et al. (Applicants specifically intend that the disclosures of all United States patent references cited herein be incorporated by reference herein in their entirety).

The polymorphisms described herein can be detected in accordance with known techniques based upon the known sequence information of the gene and the information provided herein. Novel nucleic acid sequences and proteins described herein can be isolated from human sources based upon the information provided herein or produced by other means such as site-directed mutagenesis of known or available nucleic acids, coupled as necessary with techniques for the production of recombinant proteins known in the art.

Determining the presence or absence of DNA containing a polymorphism or mutation of interest may be carried out with an oligonucleotide probe labeled with a suitable detectable group, or by means of an amplification reaction such as a polymerase chain reaction or ligase chain reaction (the product of which amplification reaction may then be detected with a labeled oligonucleotide probe or a number of other techniques). Further, the detecting step may include the step of detecting whether the subject is heterozygous or homozygous for the polymorphism of interest. Numerous different oligonucleotide probe assay formats are known which may be employed to carry out the present invention. See, e.g., U.S. Pat. No. 4,302,204 to Wahl et al.; U.S. Pat. No. 4,358,535 to Falkow et al.; U.S. Pat. No. 4,563,419 to Ranki et al.; and U.S. Pat. No. 4,994,373 to Stavrianopoulos et al. (applicants specifically intend that the disclosures of all U.S. Patent references cited herein be incorporated herein by reference).

Amplification of a selected, or target, nucleic acid sequence may be carried out by any suitable means. See generally D. Kwoh and T. Kwoh, Am. Biotechnol. Lab. 8, 14-25 (1990). Examples of suitable amplification techniques include, but are not limited to, polymerase chain reaction, ligase chain reaction, strand displacement amplification (see generally G. Walker et al., Proc. Natl. Acad. Sci. USA 89, 392-396 (1992); G. Walker et al., Nucleic Acids Res. 20, 1691-1696 (1992)), transcription-based amplification (see D. Kwoh et al., Proc. Natl. Acad Sci. USA 86, 1173-1177 (1989)), self-sustained sequence replication (or “3SR”) (see J. Guatelli et al., Proc. Natl. Acad. Sci. USA 87, 1874-1878 (1990)), the Q13 replicase system (see P. Lizardi et al., BioTechnology 6, 1197-1202 (1988)), nucleic acid sequence-based amplification (or “NASBA”) (see R. Lewis, Genetic Engineering News 12 (9), 1 (1992)), the repair chain reaction (or “RCR”) (see R. Lewis, supra), and boomerang DNA amplification (or “BDA”) (see R. Lewis, supra).

DNA amplification techniques such as the foregoing can involve the use of a probe, a pair of probes, or two pairs of probes which specifically bind to DNA containing the polymorphism of interest, but do not bind to DNA that does not contain the polymorphism of interest under the same hybridization conditions, and which serve as the primer or primers for the amplification of the DNA or a portion thereof in the amplification reaction. Such probes are sometimes referred to as amplification probes or primers herein.

In general, an oligonucleotide probe which is used to detect DNA containing a polymorphism or mutation of interest is an oligonucleotide probe which binds to DNA encoding that mutation or polymorphism, but does not bind to DNA that does not contain the mutation or polymorphism under the same hybridization conditions. The oligonucleotide probe is labeled with a suitable detectable group, such as those set forth below in connection with antibodies. Such probes are sometimes referred to as detection probes or primers herein.

Probes and primers, including those for either amplification and/or protection, are nucleotides (including naturally occurring nucleotides such as DNA and synthetic and/or modified nucleotides) are any suitable length, but are typically from 5, 6, or 8 nucleotides in length up to 40, 50 or 60 nucleotides in length, or more. Such probes and or primers may be immobilized on or coupled to a solid support such as a bead, chip, pin, or microtiter plate well in accordance with known techniques, and/or coupled to or labeled with a detectable group such as a fluorescent compound, a chemiluminescent compound, a radioactive element, or an enzyme in accordance with known techniques.

Polymerase chain reaction (PCR) may be carried out in accordance with known techniques. See, e.g., U.S. Pat. Nos. 4,683,195; 4,683,202; 4,800,159; and 4,965,188. In general, PCR involves, first, treating a nucleic acid sample (e.g., in the presence of a heat stable DNA polymerase) with one oligonucleotide primer for each strand of the specific sequence to be detected under hybridizing conditions so that an extension product of each primer is synthesized which is complementary to each nucleic acid strand, with the primers sufficiently complementary to each strand of the specific sequence to hybridize therewith so that the extension product synthesized from each primer, when it is separated from its complement, can serve as a template for synthesis of the extension product of the other primer, and then treating the sample under denaturing conditions to separate the primer extension products from their templates if the sequence or sequences to be detected are present. These steps are cyclically repeated until the desired degree of amplification is obtained. Detection of the amplified sequence may be carried out by adding to the reaction product an oligonucleotide probe capable of hybridizing to the reaction product (e.g., an oligonucleotide probe of the present invention), the probe carrying a detectable label, and then detecting the label in accordance with known techniques, or by direct visualization on a gel. When PCR conditions allow for amplification of all ApoE allelic types, the types can be distinguished by hybridization with allelic specific probe, by restriction endonuclease digestion, by electrophoresis on denaturing gradient gels, or other techniques.

Ligase chain reaction (LCR) is also carried out in accordance with known techniques. See, e.g., R. Weiss, Science 254, 1292 (1991). In general, the reaction is carried out with two pairs of oligonucleotide probes: one pair binds to one strand of the sequence to be detected; the other pair binds to the other strand of the sequence to be detected. Each pair together completely overlaps the strand to which it corresponds. The reaction is carried out by, first, denaturing (e.g., separating) the strands of the sequence to be detected, then reacting the strands with the two pairs of oligonucleotide probes in the presence of a heat stable ligase so that each pair of oligonucleotide probes is ligated together, then separating the reaction product, and then cyclically repeating the process until the sequence has been amplified to the desired degree. Detection may then be carried out in like manner as described above with respect to PCR.

It will be readily appreciated that the detecting steps described herein may be carried out directly or indirectly. For example, a polymorphism or mutation could be detected by measuring by digestion with restriction enzymes, detection of markers that are linked to the mutation or polymorphism, etc.

Kits useful for carrying out the methods of the present invention will, in general, comprise one or more oligonucleotide probes and other reagents for carrying out the methods as described above, such as restriction enzymes, optionally packaged with suitable instructions for carrying out the methods. The kits may also include containers for housing elements included therein. Such containers include, but are not limited to, vials, ampoules, tubes, capsules, bottles, syringes, and bags.

The new polymorphisms described herein provide novel nucleic acids encoding the human gene, along with probes such as described above that bind selectively thereto.

Another aspect of the invention involves the use of a combination of biomarkers to reduce the frequency of false positives or false negatives by the use of any one biomarker alone. For example, where a subject is tested for δ-catenin in the manner described herein, that subject may also be tested for the presence of another biomarker. For example, the presence of at least two biomarkers indicates that the subject is more likely to be afflicted with cancer than if only one biomarker is found in that subject; the absence of at least two biomarkers indicates the subject is more likely to be free of cancer than if only one biomarker is found in that subject; the presence of one biomarker in a subject indicates the subject is more likely to be afflicted with cancer than if the subject is found to be free of another, different, biomarker, etc. Particular biomarkers that may be used in combination with the methods of testing for δ-catenin as described herein include tight junction and adherens junction proteins such as claudin and cadherin (particularly E-cadherin), prostate specific antigen, and p120 (particularly p120^{ctn}).

The presence or absence of other cancer biomarkers may be detected in accordance with known techniques. Methods of detecting, diagnosing or screening for cancer (particularly prostate cancer).by detecting the presence of prostate specific antigen (PSA) are known and described in, among other things, U.S. Pat. Nos. 5,614,372; 5,840,501; 6,300,088; 6,361,955; 6,383,759; 6,423,503; and 6,482,599. Methods of detecting, diagnosing or screening for cancer (including prostate cancer) by detecting cadherins such as E-, OB-, N- and T-cadherin (particularly E-cadherin) are known and described in, among other things, U.S. Pat. Nos. 5,597,725; 5,811,518; 5,997,866; 6,682,901; and 6,723,320. Methods of detecting, diagnosing or screening for cancer by detecting p120 (including p120^{ctn}) are known and described in, among other things, U.S. Pat. No. 4,902,615. The disclosures of all patent references cited herein are to be incorporated by reference herein in their entirety.

The present invention is illustrated in greater detail in the following non-limiting Examples.

### EXAMPLES

### Delta-Catenin Gene (CTNND2)

### Mutations or Polymorphisms in Prostate Cancer

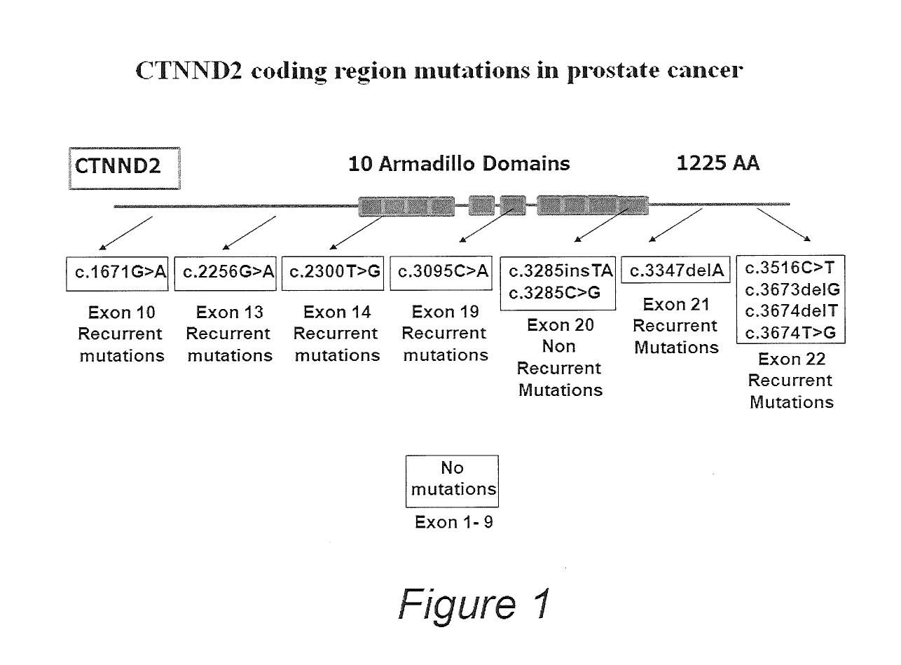

δ-Catenin (NPRAP/Neurojungin; gene designation: CTNND2, NCBI RefSeq Accession No. NM_{—}001332.2 (mRNA), GenBank Accession No. EU350953 (complete cds), not to be confused with p120^{ctn} with gene designation CTNND1) is a unique β-catenin/armadillo domain-containing p120^{ctn} subfamily protein (NCBI RefSeq Accession No. NP_{—}001323.1) in that it is primarily expressed in the central nervous system. However, δ-catenin is upregulated in over 80% of human prostatic adenocarcinomas although how its expression is regulated is currently unclear. Analyses of δ-catenin CpG islands in the promoter region in benign and prostate cancer specimens revealed no significant hyper- or hypomethylation. Real time PCR analyses found no evidence of gene amplification in δ-catenin. However, we have observed an increased incidence of mutations in the coding region or 3′ untranslated region of the δ-catenin gene when compared to that of benign prostatic tissue specimens as well as to that of peripheral blood samples of normal control subjects. Mutations are identified in TABLE 1 and FIG. 1 below.

Regarding the results set forth in Table 1, at present it is unclear where the fusion begins before exon 22. This is because there is a large intron before exon 22. It is known where the fusion ends: In the three cases that show gene fusion, they end at 10 nucleotides 5′ from exon 22.

It has also been found that that ectopic expressed delta-catenin, exon 20-22 area sequences are chaotic upon growth in vitro for a period of time. Control sequences from exon 20-22 do not exhibit this behavior. This indicated that tumor microenvironment presses on delta-catenin to eliminate certain sequences at least up to exon 20-22. Considering the time needed for the mutations to accumulate, this result indicated that cancer may have progressed further from the initial stage.

In the commercial case and case # 7288, mutations occur in exon 14 and 17. Although we have not confirmed mutations at these two exons in ectopic expressed delta-catenin, our Western blot showed that protein of the same size prediction is detected, indicating that mutations can be induced at exon 14 and 17. We believe that this area is one of the most prone locations for mutations that lead to ultimate functional alterations.

Most of the mutations identified lead to pre-mature termination, i.e. to expression of a truncated delta-catenin missing carboxyl terminus of various sizes. If delta-catenin translation terminates around exons 14 and 15, then any further mutations at exon 20-22 do not matter functionally. However, the frequency and the extent of mutations on the carboxyl terminus of delta-catenin strongly suggested that the selection pressure on the regions beginning at exon 14 is very high.

Further examples of mutations in prostate cancer samples observed within the CTNND2 coding regions are depicted in FIG. 1. Recurrent mutations are seen in Exons 10, 13, 14, 19, 21 and 22. More particularly, noted mutations include changes to: a replacement with A at nucleotide (nt) 1671 (in reference to the A of the ATG start codon in the CTNND2 mRNA, Accession No. NM_001332.2); a replacement with A at nt 2256; G at nt 2300; a replacement with A at nt 3095; an insertion of TA between nt 3285 and 3286; a replacement with G at nt 3285; a deletion of A at nt 3347; a replacement with T at nt 3516; a deletion of G at 3673; a deletion of T at nt 3674; and a replacement with G at nt 3674. These nucleotide mutations can lead to the following mutations in the amino acids of the CTNND2 protein (Accession No. NP_001323.1): I 767 to T; S 1032 to a stop codon; a frame shift at S 1095 leading to a termination in Exon 20; a frameshift at Q 1116 that adds a novel 22 amino acid peptide at the C-terminal; a frameshift resulting in W 1224 to C with removal of the original CTNND2 stop codon; and a frameshift resulting in V 1225 to G and removal of the original CTNND2 stop codon.

The foregoing is illustrative of the present invention, and is not to be construed as limiting thereof. The invention is defined by the following claims with equivalents of the claims to be included therein.

TABLE 1
Human CTNND2 exon 14, exon 15, exon 17, exon 20, exon 21, and exon 22 sequence analysis

| | | | | | |
|---|---|---|---|---|---|
| A: Case 5# | Gleason | pT2c | pN0 | pMX | |
| | 3 + 3 = 6 (with a minor | | | | |
| | popu = 4) 50 | | | | |
| B: Case 4 (4328) | Gleason 4 + 3 = 7 | 59 | pT2c | pN0 | pMX |
| C: Case 7 (6264) | Gleason 4 + 3 = 7 | pT2c | pNX | pMX | |
| D: Case 10 (7409) | Gleason 3 + 4 = 7 | pT2a | Pn0 | pMX | |

| | | | | Amino Acid | |
|---|---|---|---|---|---|
| | | Nucleotide (location | Nucleotide (location in | (in | |
| | | in reference to each | reference to mRNA | reference to | |
| | Sample Name | exon) | from ATG) | translation) | |
| exon 20 | D: Case 10 | 74 C to T | 3285 C to T | S 1095 to S | then codon structure changed and |
| | | 76, 77, insert GA | 3287, 3288 insert GA | N 1096 to R | has a stop codon TGA at 3338 in |
| | | | | | CTNND2 mRNA or amino acid |
| | | | | | 1113 |
| exon 21 | all 4 case no SNP were detected | | | | |
| exon 22 | A: Case 5# | insertion of other genes at location 10 nucleotide before exon 22 | | | |
| | | 4 miss T | 3421 miss T | S 1140 to Q | then codon structure changed and |
| | | 257 T to G | 3674 T to G | V 1225 to G | has a stop codon TGA at 3636 in |
| | | | | | CTNND2 mRNA or amino acid |
| | | | | | 1212 |
| | B: Case 4 | insertion of other genes at location 10 nucleotide before exon 22 | | | |
| | | 4 miss T | 3421 miss T | S 1140 to Q | then codon structure changed and |
| | | 257 T to G | 3674 T to G | V 1225 to G | has a stop codon TGA at 3636 in |
| | | | | | CTNND2 mRNA or amino acid |
| | | | | | 1212 |
| | C: Case 7 | insertion of other genes at location 10 nucleotide before exon 22 | | | |
| | | 4 miss T | 3421 miss T | S 1140 to Q | then codon structure changed and |
| | | 257 T to G, 258 G | 3674 T to G, 3675 G | V 1225 to G | has a stop codon TGA at 3636 in |
| | | to T | to T | | CTNND2 mRNA or amino acid |
| | | 259 T to G | 3676 T to G | stop codon | 1212 |
| | | | | change to G | |
| | D: Case 10 | | | | |
| | | 256 G to T, 257 T to | 3673 G to T, 3674 T to | V 1225 to C | |
| | | G, 258 G to T | G, 3675 G to T | | |
| | | 259 T to G, 260 miss G | 3676 T to G, 3677 miss G | stop codon | |
| | | | | changed | |
| exon 14 | a commercial case | 170 A to G, 171, 172 | 2447 A to G, | Missing | K816 will no longer be translated. |
| | | miss GA | 2448, 2449 miss GA | K816 | This frame-shift leads to stop |
| | | | | | codon in exon 15 (with 2538 |
| | | | | | nucleotide or amino acid 846 |
| | | | | | becoming a TAG stop codon) |
| | PC case 7288 | 170 miss A | 2447 miss A | K 816 to R | leads to stop codon in exon 15, |
| | | 155 G to T | 2432 G to T | K 811 to N | Stop codon at amino acid 825 |
| exon 17 | PC case 7288 | 147 A to C, | 2424 A to C, | K 811 to Q | |

* the fusion insertion before exon 22 may partly come from
1 chromosome 7: epidermal growth factor receptor isoform c precursor
2 chromosome 11: cell adhesion molecule 1 isoform 2
3 chromosome 11: neurotrimin isoform 2

1. A method for screening for risk of cancer in a subject comprising the steps of:
detecting the presence or absence of at least one mutation in the delta-catenin coding region or 3′ untranslated region in a biological sample from said subject;
the presence of said mutation or an increased frequency of mutation indicating said subject is afflicted with or at least at risk of developing cancer.

2. The method of claim 1, wherein said at least one mutation is located in delta catenin exon 10, exon 13, exon 14, exon 15, exon 16, exon 17, exon 18, exon 19, exon 20, exon 21, or exon 22 or introns before, between, or after these exons.

3. The method of claim 1, wherein said at least one mutation results in a truncated delta-catenin protein having a deleted carboxy terminus or resulting novel amino acids.

4. The method of claim 1, wherein said at least one mutation is selected from the group consisting of nucleotide mutations (in reference to mRNA from ATG):
1671 G to A;
2256 G to A;
2300 T to G;
3095 C to A;
3285 insert TA;
3285 C to G;
3347 delete A;
3516 C to T;
3673 delete G;
3674 delete T; and
3674 T to G
or amino acid mutations (in reference to translation):
Q 557 to Q;
G 752 to G:
I 767 to T;
S 1032 to Termination
S 1095 to S; Frame shift, leading to termination in exon 20.
Q 1116 to Q; Frame shift, leading to a 22 amino acid novel peptide,
F 1172 to F;
W 1224 to C; Destroy stop codon; and
V 1225 to G; Destroy stop codon.

5. The method of claim 1, wherein said subject is a human.

6. The method of claim 1, wherein said subject is male.

7. The method of claim 1, wherein said subject is female.

8. The method of claim 1, wherein said cancer is lung, breast, colon, prostate, esophageal, ovarian, pancreatic, adrenal, skin cancer or leukemia.

9. The method of claim 1, wherein said subject is male, and said cancer is prostate cancer.

10. The method of claim 1, further comprising the step of detecting the presence of a cadherin, prostate specific antigen, and/or p120 cancer biomarker in said subject,
the presence of at least one mutation in the delta-catenin gene-coding region or 3′ untranslated region in a biological sample from said subject; the presence of said mutation or an increased frequency of mutation indicating said subject is afflicted with or at least at risk of developing cancer.

11. The method of claim 1, wherein said detecting step comprises: (i) amplifying nucleic acid of said subject in said biological sample to produce an amplification product, and then (ii) detecting the presence or absence of at least one mutation in said amplification product (ii).

12. The method of claim 1, wherein said detecting step is carried out by polymerase chain reaction (PCR) and/or Single Strand Conformation Polymorphism (SSCP) followed by sequencing.

13. (canceled)

14. A kit comprising a means of detecting the presence or absence of mutation in the delta-catenin gene coding region or 3′ untranslated region in a biological sample from a subject for carrying out a method of claim 1, said kit optionally including instructions for carrying out said method.
